(19) United States
(12) Patent Application Publication
(10) Pub. No.: US 2018/0015028 A1
(43) Pub. Date: Jan. 18, 2018

(54) ANTI-WRINKLING PAD WITH ACTIVATOR SUBSTANCE

(71) Applicant: Renita LLC, New York, NY (US)

(72) Inventors: Giselle Howard, New York, NY (US); Lorrie D. King, New York, NY (US); Emma Phillips, New Canaan, CT (US)

(21) Appl. No.: 15/649,025

(22) Filed: Jul. 13, 2017

Related U.S. Application Data
(60) Provisional application No. 62/362,193, filed on Jul. 14, 2016.

(51) Int. Cl. A61K 8/99; A61K 8/44; A61K 8/81; A61K 8/73; A61K 8/04; A61K 8/49; A61K 8/42; A61K 8/362; A61K 8/34; A61K 8/19; A61Q 19/08; A61K 8/60

(52) U.S. Cl. CPC A61K 8/99; A61Q 19/08; A61K 8/44; A61K 8/8194; A61K 8/735; A61K 8/602; A61K 8/498; A61K 8/4913; A61K 8/9789; A61K 8/42; A61K 8/362; A61K 8/345; A61K 8/34; A61K 8/19; A61K 8/042; A61K 2800/87

(57) ABSTRACT

Some embodiments may provide a revolutionary cosmetic product to reduce the appearance of fine lines and/or wrinkles in a person's skin. In particular, an anti-wrinkle system may include a substantially flat silicone pad to be affixed to a person's skin (e.g., in an area around the person's chest, torso, neck, face, hands, backside, knees, etc.) and an activator substance to be applied between the silicone pad and the person's skin, including a humectant and a ferment in a suitable carrier. The activator substance might be associated with, for example, a gel, a cream, a lotion, a serum, and/or a mist. According to some embodiments, the anti-wrinkle medical grade silicone pad uses a mixture of ingredients harnessing them to represent a breakthrough, game-changing advance in the field of cosmetics that enhances the benefits of the anti-wrinkle medical grade silicone pad through additional wrinkle reduction beyond the anti-wrinkly pad alone.

### CROSS-REFERENCE TO RELATED APPLICATION

The present application claims the benefit of U.S. patent application Ser. No. 62/362,193 entitled “Anti-Wrinkling Pad With Activator Substance” filed on Jul. 14, 2016. The entire content of that application is incorporated herein by reference.

### BACKGROUND

This invention relates generally to an apparatus that reduces the appearance of fine lines and/or wrinkles on a person's skin, and, more specifically, to a silicone pad and activator substance.

An increasing amount of imperfections, such as fine lines and/or wrinkles, may appear on a person's skin as he or she ages. Many people prefer to reduce the appearance of such imperfections to achieve a younger and more healthy image. In general, fine lines and/or wrinkles are folds, ridges or creases in a person's skin. In some cases, wrinkles may appear as a result of aging processes such as glycation (which may damage collagen and elastin associated with protein fibers that keep skin firm and elastic), loss of body mass, etc. In addition to aging, wrinkles may be caused by habitual facial expressions, sun damage, smoking, poor hydration, etc., any of which might make a person uncomfortable with his or her appearance. Accordingly, it would be desirable to provide a system which can effectively reduce the appearance of fine lines and/or wrinkles on a person's skin by being applied over a period of time, such as while he or she is sleeping or for a few hours during the day.

### SUMMARY OF THE INVENTION

According to some embodiments, apparatus, methods, and means may be provided to facilitate the reduction of wrinkles by being applied over a period of time, such as while a person sleeps or for a few hours during the day. In some embodiments, an anti-wrinkle system may include a substantially flat silicone pad to be affixed to a person's skin (e.g., in an area around the person's chest, torso, neck, face, hands, backside, knees, etc.) and an activator substance to be applied between the silicone pad and the person's skin, including a humectant and a ferment in a suitable carrier. The activator substance might be associated with, for example, a gel, a cream, a lotion, a serum, and/or a mist. According to some embodiments, the anti-wrinkle pad uses a mixture of ingredients harnessing them to represent a breakthrough, game-changing advance in the field of cosmetics.

A technical effect of some embodiments of the invention is a greatly improved way to reduce the appearance of fine lines and/or wrinkles around a person's chest, torso, neck, face, hands, backside, knees, etc. over a period of time, such as during a person's sleep. With these and other advantages and features that will become hereinafter apparent, a more complete understanding of the nature of the invention can be obtained by referring to the following detailed description and to the drawings appended hereto.

### BRIEF DESCRIPTION OF THE DRAWINGS

FIG. 1 is an overview of a wrinkle reduction environment according to some embodiments of the present invention.

FIG. 2 is a top perspective view of an anti-wrinkle pad according to some embodiments of the present invention.

FIG. 3 is a top plan view of an anti-wrinkle pad according to some embodiments of the present invention.

FIG. 4 is a side plan view of the cross-section 4-4 illustrated in FIG. 3 in accordance with some embodiments.

FIG. 5 is a side plan view of the cross-section 5-5 illustrated in FIG. 3 in accordance with some embodiments.

FIGS. 6 through 8 illustrate the application of an anti-wrinkle pad to a person's skin in accordance with some embodiments.

FIG. 9 illustrates a method of use of an anti-wrinkle pad by a person according to some embodiments of the present invention.

FIGS. 10 through 13 illustrate various wrinkle reduction embodiments in accordance with some embodiments.

### DETAILED DESCRIPTION

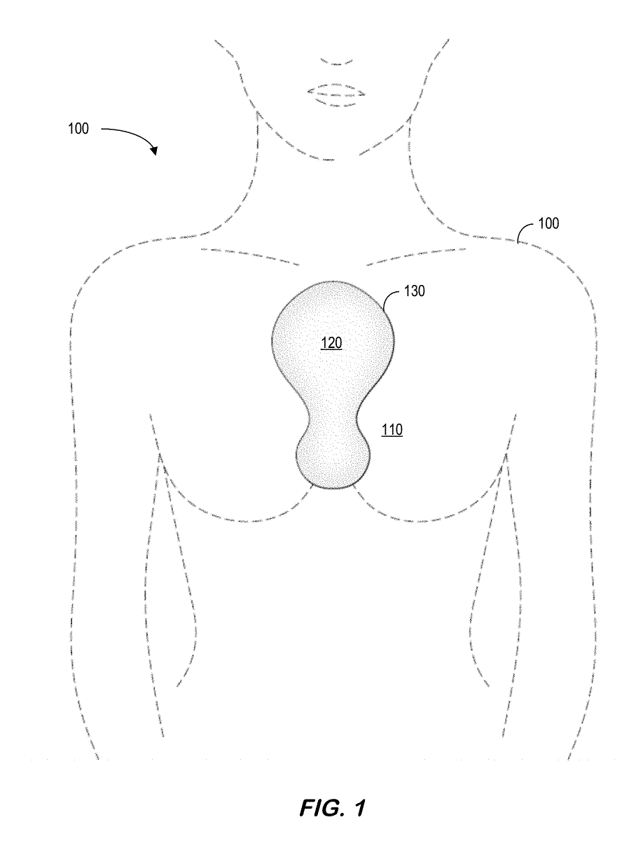

FIG. 1 is an overview of a wrinkle reduction environment 100 according to some embodiments of the present invention. In particular, a person 100 has adhered an anti-wrinkle system, including a silicone pad 120, to an area of skin 110 around the chest, torso, neck, face, hands, backside, knees, etc. According to some embodiments, the person 100 (or some other party) may also apply an activator substance (not shown in FIG. 1) between the silicone pad 120 and the area of skin 110. Note that the shape of the silicone pad 120 and the area of skin 110 illustrated in FIG. 1 are only examples, and embodiments might be associated with any other shape for the silicone pad 120 and area of skin 110. Note that the face, neck, and décolleté may age faster than other parts of the body due to having thinner skin, less collage density, and a thinner dermis. These may all contribute to increased skin wrinkling that may be addressed by embodiments described herein.

The silicone pad 120 may have, according to some embodiments, an upper surface (seen in FIG. 1), a lower surface, a perimeter edge 130, and the system (e.g., the silicone pad 120 and the activator substance) may have one or more characteristics (e.g., chemicals, shape, elasticity, etc.) to help reduce the appearance of wrinkles in the area 110 around the person's chest, torso, neck, face, hands, backside, knees, etc. In this way, a person may comfortably wear the pad for an extended period of time (e.g., overnight while the person sleeps or for a few hours during the day) to reduce the appearance of fine lines, wrinkles and/or other skin imperfections. According to some embodiments, the silicone pad 120 is formed of a mixture of ingredients harnessing them to represent a clear advance over the traditional approaches taken with cosmetics to reduce the appearance of fine lines and/or wrinkles on a person's skin. Note that embodiments described herein may help reduce dehydration, roughness, wrinkling, crinkling, creasing, dullness, dryness, and/or inflammation associated with a person's skin.

According to some embodiments, an infused substance is provided within the silicone pad 120 or an adhesive substrate. The infused substance may, for example, be transferred to the person's skin 110 over a period of time to provide a beneficial result. The infused substance could be, for example, a vitamin or any other chemical (e.g., natural, organic, synthetic, etc.) that might improve or otherwise provide advantages to the skin 110 of the person wearing the anti-wrinkle pad. As used herein, the term “infused” might refer to, for example, a microencapsulated substance.

Some embodiments described herein are directed to a personal care system or composition comprising an activator and a silicone pad. The activator may be comprised of a “humectant” and a “ferment” in a suitable carrier for use under a silicone based-pad. As used herein, the term “humectant” may refer to a substance that helps retain water. It may attract and retain moisture via absorption, drawing the water vapor into and/or beneath the organism/object's surface. In skincare particularly, humectants may penetrate the upper skin layers and attract and bind moisture, thus making the upper layers more hydrated. Examples of humectants useful in skincare include glycerin, urea and Sodium PCA. One particularly suitable humectant for the activator gel is hydroxyethyl urea.

As used herein, the term “ferment” may refer to an end product of a fermentation process that occurs due to the bulk growth of microorganisms on a growth medium, often with the goal of producing a specific chemical product. The microorganisms utilized in the process of fermentation may be used in aerobic or anaerobic environments. Some examples of microorganisms useful in fermentation include yeasts and bacteria. A specifically useful bacterium, Bacillus, is useful for producing ferments beneficial to skin. Bacillus bacteria ferments may be further refined in to filtrates by removing solids from the bulk via a filtering process.

Note that the activator substance may be developed into many forms that would be useful for skincare. Examples of suitable forms include, but are not limited to, gels, creams, lotions, serums and mists. They may be applied directly to the silicone pad or applied directly to the skin prior to covering with the silicone pad. The following are non-limiting, exemplary examples of some aforementioned forms.

According to some embodiments, the activator substance is associated with an activator gel. Table I identifies some International Nomenclature of Cosmetic Ingredients (“INCI”) elements that might be associated with the activator gel:

TABLE I
Activator Gel

| INCI | Purpose | Supplier |
|---|---|---|
| Water | Diluent | N/A |
| Carbomer | Thickener | Lubrizol or Others |
| Bacillus Ferment AND | Humectant/ | IPS Labs |
| Hydroxyethyl urea | Ferment Blend | or Others |
| Phenoxyethanol AND | Preservative | Shulke & Mayr |
| Ethylhexylglycerin | | or Others |
| Citric Acid/NaOH | | |

According to this embodiment, water and Carbomer might be added under vigorous mixing until homogenous. A Bacillus ferment and Hydroxyethyl Urea blend might then be added under vigorous mixing. Phenoxyethanol and Ethylhexylglycerin might then be added under vigorous mixing. A pH adjustment may be performed to adjust the pH to between 5.6 and 6.2 with citric acid under light mixing until a clear, thick homogenous gel is formed.

Note that the ingredients in Table I are provided only as an example, and an activator gel might be formed in any number of different ways. For example, Table II identifies some INCI elements that might be associated with another type of activator gel:

TABLE II
Activator Gel

| INCI | Purpose | Supplier |
|---|---|---|
| Water | Diluent | N/A |
| Carbomer | Thickener | Lubrizol or Others |
| Xylytylglucoside | Humectants | Seppic or Others |
| AND anhydroxylitol | | |
| AND xylitol | | |
| Hydrolyzed Hyaluronic Acid | Humectants | EVONIK or Others |
| Lactococcus Ferment Extract AND | Ferment and | BASF or Others |
| Propanediol AND Sodium Cocoyl | Stabilizers | |
| Glutamate AND Caprylyl | | |
| Glycol AND Ethylhexylglycerin | | |
| Phenoxyethanol AND | Preservative | Shulke & Mayr |
| Ethylhexylglycerin | | or Others |
| Chlorphenesin | Preservative | BASF or Others |
| Citric Acid/NaOH | | |

According to this embodiment, water and Carbomer might be added under vigorous mixing until homogenous. All the other components, except Citric Acid/NaOH might then be added under vigorous mixing. A pH adjustment may be performed to adjust the pH to between 5.5 and 6.5 with citric acid and/or Sodium Hydroxide under light mixing until a thick homogenous gel is formed.

According to some embodiments, the activator substance is associated with an activator spray. Table III identifies some INCI elements that might be associated with the activator spray:

TABLE III
Activator Spray

| INCI | Purpose | Supplier |
|---|---|---|
| Water | Diluent | N/A |
| Glycerin | Humectant | Acme-Hardesty |
| | | or Others |
| Bacillus Ferment | Ferment | IPS Labs or Others |
| Phenoxyethanol (and) | Preservative | Shulke & Mayr |
| Ethylhexylglycerin | | or Others |
| Citric Acid/NaOH | pH Adjuster | |

According to this embodiments, water and glycerin may be combined under vigorous mixing until homogenous. A Bacillus ferment may then be added under vigorous mixing. Phenoxyethanol and Ethylhexylglycerin may then be added under vigorous mixing. A pH adjustment may then be performed to adjust the pH to between 5.6 and 6.2 with citric acid or sodium hydroxide under light mixing until a clear, homogenous solution is formed.

According to some embodiments, the activator substance is associated with an activator serum. Table IV identifies some INCI elements that might be associated with the activator serum:

TABLE IV
Activator Serum

| INCI | Purpose | Supplier |
|---|---|---|
| Water | Diluent | N/A |
| Carbomer | Thickener | Lubrizol or Others |
| Sodium PCA | Humectant | Ajinomoto |
| (and) Water | | or Others |
| Bacillus Ferment | Ferment | IPS Labs or Others |
| Phenoxyethanol | Preservative | Lonza or Others |
| (and) Chlorphenesin | | |
| (and) Caprylyl Glycol | | |
| Citric Acid/NaOH | pH Adjuster | |

According to this embodiment, water, Carbomer and sodium PCA may be combined under vigorous mixing until homogenous. Glycerin and a Bacillus ferment may then be added under vigorous mixing. Phenoxyethanol, Chlorphenesin, Caprylyl Glycol may then be added under vigorous mixing. A pH adjustment may then be performed to adjust the pH to between 5.6 and 6.2 with citric acid under light mixing until a clear, thin homogenous gel is formed.

According to some embodiments, the activator substance is associated with an activator lotion. Table V identifies some INCI elements that might be associated with the activator lotion:

TABLE V
Activator Lotion

| INCI | Supplier |
|---|---|
| Water | N/A |
| Carbomer | Lubrizol or Others |
| Bacillus Ferment | IPS Labs or Others |
| (and) Hydroxyethyl | |
| Urea | |
| Steareth-2 (and) | Croda or Others |
| Steareth-21 | |
| Capric/Caprylic | Protameen or |
| Triglycerides | Others |
| Shea Butter | Premier Specailties |
| | or Others |
| Cetearyl Alcohol | Croda or Others |
| Phenoxyethanol (and) | Lonza or Others |
| Chlorphenesin (and) | |
| Caprylyl Glycol | |
| Citric Acid/NaOH | |
| Water | |

This embodiment might be associated with a two phase process. In a first phase, water, Carbomer, a Bacillus ferment, Hydroxyethyl Urea may be added to a vessel and blended under mixing until homogenous. The mixture may then be heated to 60° Celsius (“C.”). In a second phase, Steareth-2, Steareth-21, Capric/Caprylic Triglycerides, Shea Butter, and Cetearyl alcohol may be added and heated to 60° C. under mixing until homogenous. When both phases are at 60° C., the phases may be under vigorous mixing and remove from heat. When the batch temperature reaches 40° C., Phenoxyethanol, Chlorphenesin, Caprylyl Glycol may be added under mixing. A pH adjustment process may then be performed to adjust the pH to between 5.6 and 6.2 with citric acid under light mixing until an opaque, thick, homogenous cream is formed.

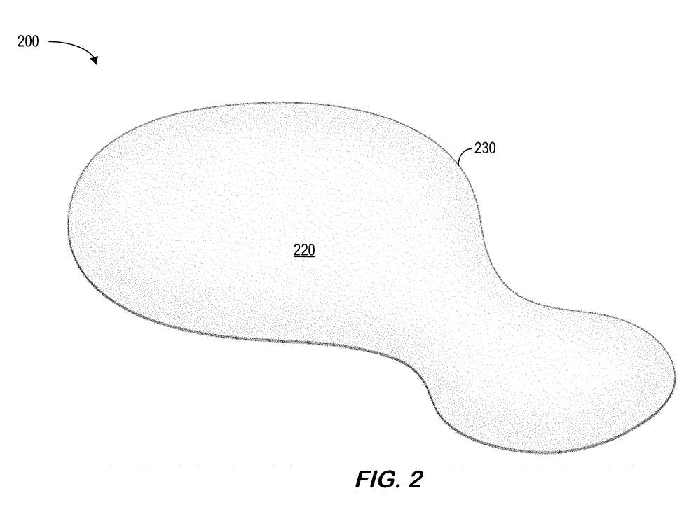

FIG. 2 is a top perspective view of an anti-wrinkle pad 200 according to some embodiments of the present invention. The pad 200 includes an anti-wrinkle portion 220 having a perimeter edge 230. According to some embodiments, the perimeter edge 230 is formed using smooth, tapered curves to reduce existence of sharp angles (e.g., right angles). Such an approach may, for example, help avoid the presence of marks on a person's skin after the pad 200 is removed after a period of time (e.g., overnight or for a few hours during the day). Note that any of the embodiments described herein might utilize non-tapered edges instead.

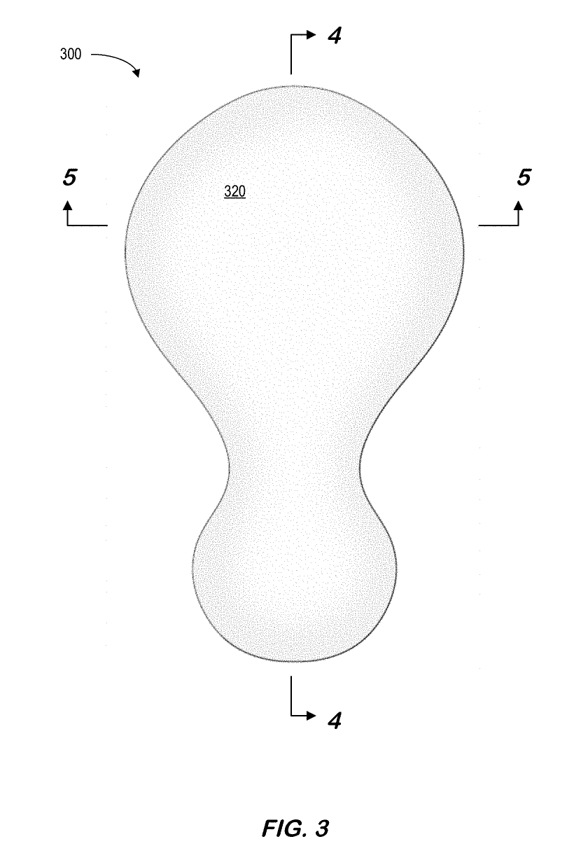

FIG. 3 is a top plan view of an anti-wrinkle pad 300 according to some embodiments of the present invention. As before, the pad 300 includes an anti-wrinkle portion 320 having a perimeter edge. According to some embodiments, the pad 300 has a height (from the top of FIG. 3 to the bottom of FIG. 3) of substantially 200 millimeters. Moreover, according to some embodiments, the pad 300 has a maximum width (from the left of FIG. 3 to the right of FIG. 3) proximate to the top of the pad of approximately 105 millimeters. Moreover, according to some embodiments the pad 300 has a width proximate to the bottom of the pad of approximately 69 millimeters. By having the top of the pad 300 wider as compared to the bottom of the pad 300, a person may more comfortably affix the pad 300 to an area of skin located around a person's chest, torso, neck, face, hands, backside, knees, etc.

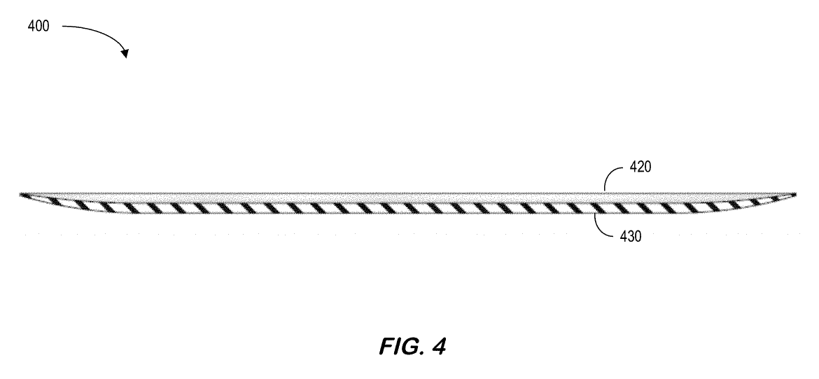

FIG. 4 is a side plan view of the cross-section 4-4 illustrated in FIG. 3 in accordance with some embodiments. The pad 400 includes the anti-wrinkle portion 420 and a substantially flat adhesive substrate 430 affixed to the lower surface of the anti-wrinkle portion 420. The adhesive substrate 430 may, according to some embodiments, be adapted to adhere the anti-wrinkle portion 420 to the person's skin, and the anti-wrinkle portion 420 and adhesive substrate 430 may be arranged in a parallel planer manner in relation to each other. Moreover, according to some embodiments, the adhesive substrate 430 may comprise polydimethylsiloxane. According to some embodiments, both the anti-wrinkle portion 420 and/or the adhesive substrate 430 may provide benefits, including anti-wrinkle characteristics. The anti-wrinkle portion 420 may, for example, provide mechanical resistance to prevent skin folding, the adhesive substrate 430 may provide a barrier to skin dehydration, exfoliation (when removed), and help smooth ridges and/or creases in skin (when removed), etc.

According to some embodiments, the anti-wrinkle portion 420 comprises silicone. For example, the anti-wrinkle portion 420 might include methyl vinyl silicone. More particular, in some embodiments, the anti-wrinkle portion may be formed of: polydimethylsiloxane; silicone dioxide; hydroxyl-terminated silicone fluid; stearic acid; and/or other substances.

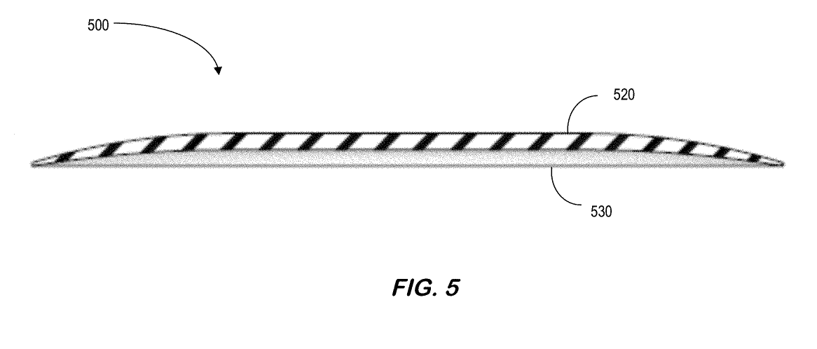

FIG. 5 is a side plan view of the cross-section 5-5 illustrated in FIG. 3 in accordance with some embodiments. According to some embodiments, the pad 500, including an anti-wrinkle portion 520 and an adhesive substrate, has an overall thickness of substantially 5 millimeters. Note that, as illustrated in FIG. 5, the anti-wrinkle portion 520 and adhesive substrate 530 each have a thickness that is largest near the center of the pad 500 and that tapers off toward the perimeter edge. According to some embodiments, the anti-wrinkle portion 520 and adhesive substrate 530 each have a thickness of substantially 2.5 millimeters near the center of the pad 500.

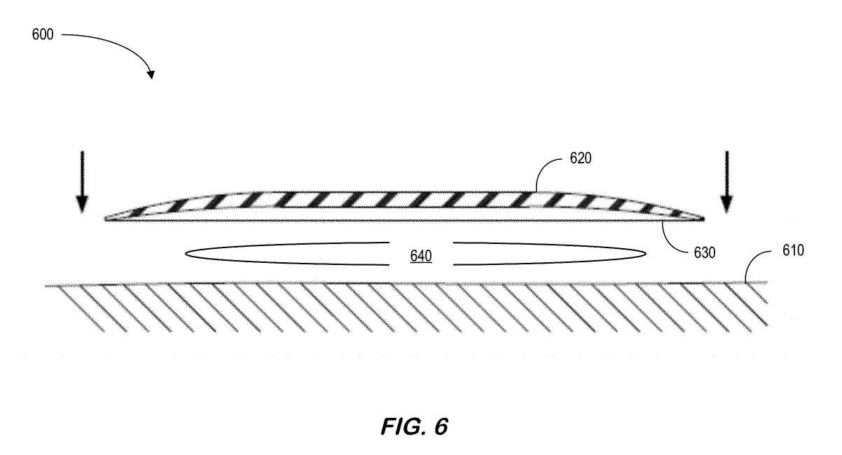

FIGS. 6 through 8 illustrate the application of an anti-wrinkle pad to a person's skin in accordance with some embodiments. As illustrated 600 in FIG. 6, an anti-wrinkle portion 620 may, according to some embodiments, be molded with a mild suction cup cross-section (e.g., via a void in the adhesive layer 630) that, when adhered to a person's skin 610, attempts to revert to an original form. Consider, for example, movement of the silicone pad, including the adhesive substrate 630, toward the person's skin 610 as illustrated by the two arrows in FIG. 6. According to some embodiments, an activator substance 640 is provided between the silicone pad and the person's skin 610. The activator substance 640 might be, for example, applied directly to the silicone pad and/or the person's skin 610.

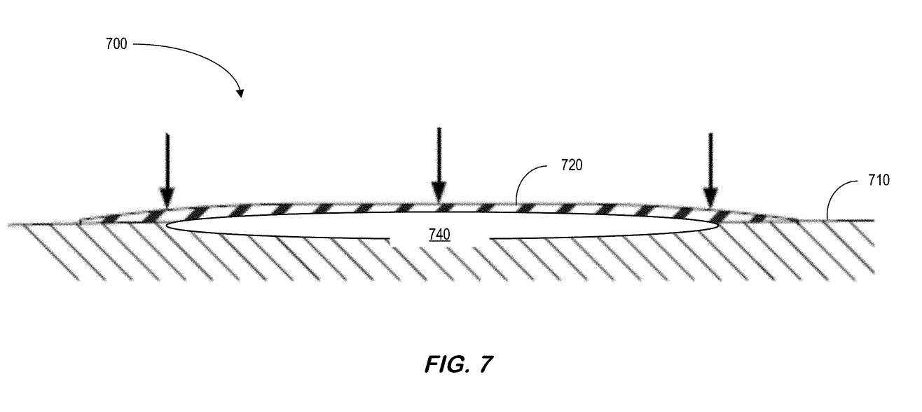

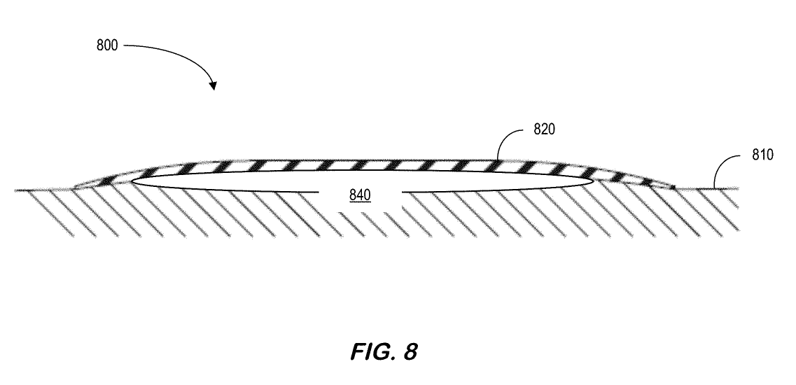

As illustrated 700 in FIG. 7, the person might apply slight pressure to the anti-wrinkle portion 720 (as illustrated by the three arrows in FIG. 7) to adhere the pad to the skin 710. Note that the activator substance 740 remains between the silicone pad and the person's skin 710. The mild suction cup-cross section of the anti-wrinkle portion 720 has deformed to follow the contours of the person's skin 710. According to some embodiments, this may create a slight tension in the person's skin 710 pulling the skin 710 upward and toward the perimeter edge of the pad. For example, FIG. 8 illustrates 800 how the person's skin 810 has moved up into the suction cup area of the anti-wrinkle portion 820. Such an effect may, according to some embodiments, promote blood flow to the area which may help reduce wrinkles and/or other imperfections in the person's skin. Note that the activator substance 840 remains between the silicone pad and the person's skin 810.

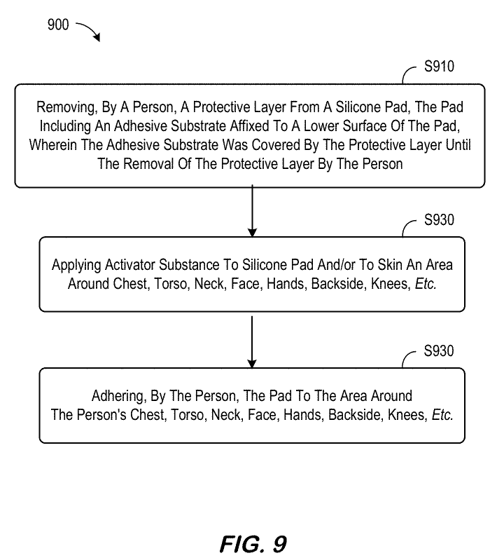

FIG. 9 illustrates a method 900 of use of an anti-wrinkle pad by a person according to some embodiments of the present invention. At S910, a person may remove a protective layer from a pad, the pad including an adhesive substrate affixed to a lower surface of the silicone pad, wherein the adhesive substrate is adapted to adhere the anti-wrinkle portion to the person's skin. Note that the anti-wrinkle portion and adhesive substrate may be arranged in a parallel planer manner in relation to each other. The protective layer might be associated with, for example, a pouch, envelope, or other packaging. According to some embodiments, the adhesive substrate comprises polydimethylsiloxane that was covered by the protective layer until the removal of the protective layer by the person. At S920, an activator substance may be applied to the silicone pad and/or the person's skin. At S930, the person may adhere the pad to an area around the person's chest, torso, neck, face, hands, backside, knees, etc. Note that any of the embodiments described herein might be associated with a single-use pad (e.g., a disposable pad). Moreover, any embodiment might instead be associated with a re-usable pad (e.g., a pad that can be applied and re-applied up to fifteen times).

Thus, embodiments may provide a substantial reduction in the appearance of fine lines and/or wrinkles around a person's chest, torso, neck, face, hands, backside, knees, etc. during sleep. As a result, consumers may quickly and easily achieve a more youthful and healthy appearance. Moreover, according to some embodiments, removal of the pad may further provide some degree of exfoliation wherein some amount of dead skin cells may be removed when the pad is removed from the skin. Still further, some embodiments may provide rehydration benefits when the pad is worn by a person. For example, silicone may provide a moisture barrier that acts as a water barrier, and as a result, the person's skin may be rehydrated (e.g., overnight). According to some embodiments, the anti-wrinkle pad includes a mixture of ingredients harnessing them to represent a revolutionary, breakthrough, and game-changing advance in the field of cosmetics.

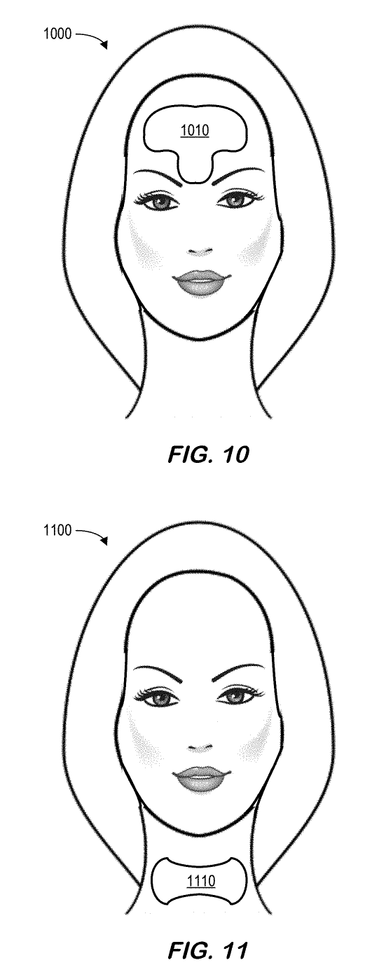

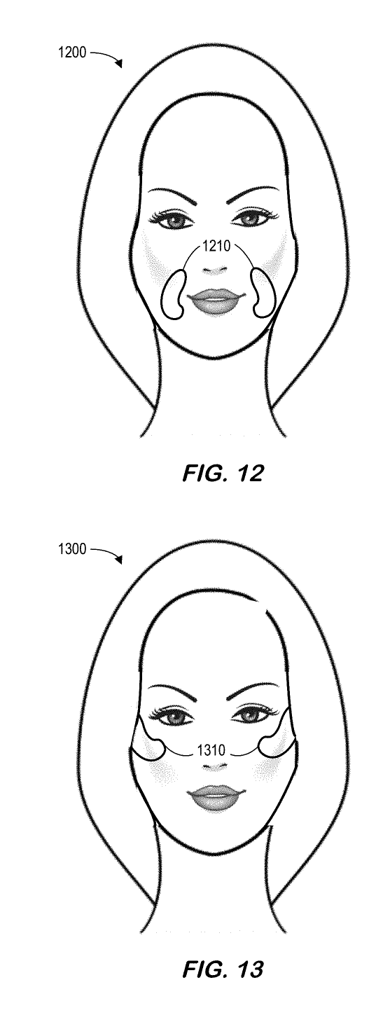

Note that the various pad shapes, sized, and application locations described herein are provided only as examples and embodiments may utilize any number of other configurations. For example, FIG. 10 illustrates a wrinkle reduction environment 1000 according to some embodiments of the present invention. In particular, a person has adhered an anti-wrinkle system, including a silicone pad 1010 (e.g., a medical grade silicone pad), to her forehead (e.g., after an activator substance was applied between the silicone pad 1010 and her skin) to provide a “brow lift.” Similarly, FIG. 11 illustrates an environment wherein a person has adhered an anti-wrinkle system, including a silicone pad 1110, to her neck to provide a “neck lift.” As another example, FIG. 12 illustrates an environment 1200 wherein a person has applied a pair of silicone pads 1210 near the corners of her mouth to provide a “smile lift.” As a final example, FIG. 13 illustrates an environment 1300 wherein a person has applied a pair of silicone pads 1310 near the outside portions of her eyes to provide, in effect, an “eye lift.”

The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although specific configurations have been described herein, note that any number of other configurations may be provided in accordance with embodiments of the present invention (e.g., shapes other than those illustrated might be used for the perimeter edge and overall shape of an anti-wrinkle pad).

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

What is claimed:

1. An anti-wrinkle system, comprising:
a substantially flat silicone pad to reduce an appearance of fine lines and/or wrinkles on a person's skin in an area onto which the silicone pad is affixed; and
an activator substance to be applied between the silicone pad and the person's skin, including:
a humectant, and
a ferment in a suitable carrier.

2. The system of claim 1, wherein the activator substance is associated with at least one of: (i) a gel, (ii) a cream, (iii) a lotion, (iv) a serum, and (v) a mist.

3. The system of claim 1, wherein the activator substance is to be applied to the person's skin before the silicone pad is affixed.

4. The system of claim 1, wherein the activator substance is to be applied to the silicone pad before being affixed to the person's skin.

5. The system of claim 1, wherein the activator substance is a gel that includes water, Carbomer, bacillus ferment, hydroxyethyl urea, phenoxyethanol, ethylhexylglycerin, citric acid, and NaOH.

6. The system of claim 1, wherein the activator substance is a gel that includes water, Carbomer, Xylytylglucoside, anhydroxylitol, xylitol, Hydrolyzed Hyaluronic Acid, Lactococcus Ferment Extract, Propanediol, Sodium Cocoyl Glutamate, Caprylyl Glycol, Ethylhexylglycerin, Phenoxyethanol, Ethylhexylglycerin, Chlorphenesin, Citric Acid, and NaOH.

7. The system of claim 1, wherein the activator substance is a spray that includes water, glycerin, bacillus ferment, phenoxyethanol, ethylhexylglycerin, citric acid, and NaOH.

8. The system of claim 1, wherein the activator substance is a serum that includes water, Carbomer, sodium PCA, bacillus ferment, phenoxyethanol, chlorphenesin, caprylyl glycol, citric acid, and NaOH.

9. The system of claim 1, wherein the activator substance is a lotion that includes: water, Carbomer, bacillus ferment, hydroxyethyl urea, Steareth-2, Steareth-21, Capric/Caprylic Triglycerides, Shea Butter, cetearyl alcohol, phenoxyethanol, chlorphenesin, caprylyl glycol, citric acid, and NaOH.

10. The system of claim 1, wherein the silicone pad is to be affixed in an area around the person's chest, torso, neck, face, hands, backside, or knees.

11. An anti-wrinkle method, comprising:
removing a protective layer from a silicone pad, the pad including an adhesive substrate affixed to a lower surface of the pad, wherein the adhesive substrate was covered by the protective layer until the removal of the protective layer;
applying activator substance to the silicone pad and/or to the skin in an area around a person's chest, torso, neck, face, hand, backside, or knee, wherein the activator substance includes a humectant and a ferment in a suitable carrier; and
adhering the pad to the area around the person's chest, torso, neck, face, hand, backside, or knee.

12. The method of claim 11, wherein the activator substance is associated with at least one of: (i) a gel, (ii) a cream, (iii) a lotion, (iv) a serum, and (v) a mist.

13. The method of claim 11, wherein the activator substance is to be applied to the person's skin before the silicone pad is affixed.

14. The method of claim 11, wherein the activator substance is to be applied to the silicone pad before being affixed to the person's skin.

15. The method of claim 11, wherein the activator substance is a gel that includes water, Carbomer, bacillus ferment, hydroxyethyl urea, phenoxyethanol, ethylhexylglycerin, citric acid, and NaOH.

16. The method of claim 11, wherein the activator substance is a gel that includes water, Carbomer, Xylytylglucoside, anhydroxylitol, xylitol, Hydrolyzed Hyaluronic Acid, Lactococcus Ferment Extract, Propanediol, Sodium Cocoyl Glutamate, Caprylyl Glycol, Ethylhexylglycerin, Phenoxyethanol, Ethylhexylglycerin, Chlorphenesin, Citric Acid, and NaOH.

17. The method of claim 11, wherein the activator substance is a spray that includes water, glycerin, bacillus ferment, phenoxyethanol, ethylhexylglycerin, citric acid, and NaOH.

18. The method of claim 11, wherein the activator substance is a serum that includes water, Carbomer, sodium PCA, bacillus ferment, phenoxyethanol, chlorphenesin, caprylyl glycol, citric acid, and NaOH.

19. The method of claim 11, wherein the activator substance is a lotion that includes: water, Carbomer, bacillus ferment, hydroxyethyl urea, Steareth-2, Steareth-21, Capric/Caprylic Triglycerides, Shea Butter, cetearyl alcohol, phenoxyethanol, chlorphenesin, caprylyl glycol, citric acid, and NaOH.
